(19) United States
(12) Patent Application Publication
(10) Pub. No.: US 2022/0009980 A1
(43) Pub. Date: Jan. 13, 2022

(54) TP5, A PEPTIDE INHIBITOR OF ABERRANT AND HYPERACTIVE CDK5/P25 AS TREATMENT FOR CANCER

(71) Applicant: The United States of America,as represented by the Secretary,Department of Health and Human Services, Bethesda, MD (US)

(72) Inventors: Zhengping Zhuang, Bethesda, MD (US); Emeline Tabouret, Marseille (FR); Herui Wang, McLean, VA (US); Harish Pant, Rockville, MD (US); Niranjana D. Amin, Clarksburg, MD (US)

(73) Assignee: The United States of America,as represented by the Secretary,Department of Health and Human Services, Bethesda, MD (US)

(21) Appl. No.: 17/294,011

(22) PCT Filed: Nov. 13, 2019
(86) PCT No.: PCT/US2019/061251

§ 371 Date: May 14, 2021

Related U.S. Application Data
(60) Provisional application No. 62/767,230, filed on Nov. 14, 2018.

(51) Int. Cl. C07K 14/47; A61K 31/4745; C12N 15/63

(52) U.S. Cl. CPC C07K 14/4703; A61K 31/4745; A61K 38/00; C07K 14/4738; C12N 15/63

(57) ABSTRACT

Methods of decreasing cell viability of cancer cells, increasing apoptosis of cancer cells, and treating cancer in a mammal with cancer are provided. The methods include administering (i) a polypeptide comprising an amino acid sequence with at least 95% sequence identity to the amino acid sequence of SEQ ID NO: 1, (ii) a nucleic acid molecule comprising a nucleic acid sequence encoding the polypeptide, (iii) a vector comprising the nucleic acid molecule, (iv) a recombinant cell comprising any one of (i)-(iii), and/or (v) a composition comprising any one of (i)-(iv).

### CROSS-REFERENCE TO RELATED APPLICATIONS

This patent application claims the benefit of U.S. Provisional Patent Application No. 62/767,230, filed Nov. 14, 2018, which is incorporated by reference.

### STATEMENT REGARDING FEDERALLY SPONSORED RESEARCH OR DEVELOPMENT

This invention was made with Government support under project number Z01-Z1A-BC-011773 by the National Institutes of Health, National Cancer Institute. The Government has certain rights in the invention.

### SEQUENCE LISTING

Incorporated by reference in its entirety herein is a nucleotide/amino acid sequence listing submitted concurrently herewith.

### BACKGROUND OF THE INVENTION

P5 is a small peptide the selectively inhibits aberrant and hyperactive CDK/p25 (see U.S. Pat. No. 8,597,660). The peptide was modified to facilitate passage through blood brain barrier (BBB), resulting in TP5 (see U.S. Pat. No. 8,597,660). TP5 has been used for the treatment of subjects with neurodegenerative disease, such as Alzheimer's disease (see U.S. Pat. No. 8,597,660).

### BRIEF SUMMARY OF THE INVENTION

The invention provides a method of decreasing cell viability of cancer cells comprising administering to the cancer cells one or more of the following: (i) a polypeptide comprising an amino acid sequence with at least 95% sequence identity to the amino acid sequence of SEQ ID NO: 1, (ii) a nucleic acid molecule comprising a nucleic acid sequence encoding the polypeptide, (iii) a vector comprising the nucleic acid molecule, and (iv) a composition comprising any one of (i)-(iii).

The invention also provides a method of increasing apoptosis of cancer cells comprising administering to the cancer cells one or more of the following: (i) a polypeptide comprising an amino acid sequence with at least 95% sequence identity to the amino acid sequence of SEQ ID NO: 1, (ii) a nucleic acid molecule comprising a nucleic acid sequence encoding the polypeptide, (iii) a vector comprising the nucleic acid molecule, and (iv) a composition comprising any one of (i)-(iii).

Additionally, the invention provides a method of treating cancer in a mammal with cancer comprising administering to the mammal one or more of the following: (i) a polypeptide comprising an amino acid sequence with at least 95% sequence identity to the amino acid sequence of SEQ ID NO: 1, (ii) a nucleic acid molecule comprising a nucleic acid sequence encoding the polypeptide, (iii) a vector comprising the nucleic acid molecule, (iv) a recombinant cell comprising any one of (i)-(iii), and (v) a composition comprising any one of (i)-(iv).

### BRIEF DESCRIPTION OF THE SEVERAL VIEWS OF THE DRAWINGS

FIG. 1 is a graph demonstrating the survival rate of cells of glioblastoma cell line U251 following administration of TP5 or TP5 scrambled.

FIG. 2 is an image of the number of glioblastoma colonies after administration of different amounts of TP5.

FIG. 3 is a graph demonstrating the effect of TP5 administration on early and late apoptosis of cells of glioblastoma cell line U251.

FIG. 4 is an image of the expression levels of pH2AX (or actin control) in cells of glioblastoma cell line U251 following the administration of different amounts of TP5.

FIG. 5 is a graph demonstrating glioblastoma tumor volume in an orthotopic mouse model following administration of 100 μM or 300 μM of TP5.

FIG. 6 is a graph demonstrating the survival rate of cells of colorectal cancer cell line HT29 following administration of TP5 or TP5 scrambled.

FIG. 7 is an image of the number of colorectal cancer colonies after administration of different amounts of TP5.

FIG. 8 is an image of the expression levels of pH2AX (or actin control) in cells of colorectal cancer cell line HT29 following the administration of different amounts of TP5.

FIG. 9 is a graph demonstration colorectal tumor volume in a subcutaneous colorectal cancer mouse model following administration of TP5 alone, chemotherapy alone (Sn38; 7-ethyl-10-hydroxycamptothecin), or TP5+Sn38.

### DETAILED DESCRIPTION OF THE INVENTION

The inventors discovered that using TP5 reduces or inhibits one or more symptoms associated with cancer, such as glioblastoma or colorectal cancer. For example, cell viability is decreased and apoptosis is increased in cancer cells, while the proliferation rate and tumor volume are decreased. Based on these observations, methods of treatment to reduce or eliminate one or more symptoms or signs associated with cancer are disclosed.

For example, the invention provides a method of decreasing cell viability of cancer cells comprising administering to the cancer cells one or more of the following: (i) a polypeptide comprising an amino acid sequence with at least 95% sequence identity to the amino acid sequence of SEQ ID NO: 1, (ii) a nucleic acid molecule comprising a nucleic acid sequence encoding the polypeptide, (iii) a vector comprising the nucleic acid molecule, and (iv) a composition comprising any one of (i)-(iii).

The invention also provides a method of increasing apoptosis of cancer cells comprising administering to the cancer cells one or more of the following: (i) a polypeptide comprising an amino acid sequence with at least 95% sequence identity to the amino acid sequence of SEQ ID NO: 1, (ii) a nucleic acid molecule comprising a nucleic acid sequence encoding the polypeptide, (iii) a vector comprising the nucleic acid molecule, and (iv) a composition comprising any one of (i)-(iii).

Moreover, the invention provides a method of treating cancer in a mammal with cancer comprising administering to the mammal (i) a polypeptide comprising an amino acid sequence with at least 95% sequence identity to the amino acid sequence of SEQ ID NO: 1, (ii) a nucleic acid molecule comprising a nucleic acid sequence encoding the polypeptide, (iii) a vector comprising the nucleic acid molecule, (iv) a recombinant cell comprising any one of (i)-(iii), and (v) a composition comprising any one of (i)-(iv).

In some examples, the methods of use can include selecting a mammal (e.g., human subject) in need of treatment (i.e., a mammal that has cancer or is at risk of developing cancer). For example, studies can be performed to identify a mammal as being afflicted with cancer, including, but not limited to, glioblastoma and colorectal cancer. Methods of detecting cancer are known to those of skill in the art.

Non-limiting examples of specific types of cancers include cancer of the head and neck, eye, skin, mouth, throat, esophagus, chest, bone, lung, colon, sigmoid, rectum, stomach, prostate, breast, ovaries, kidney, liver, pancreas, brain, intestine, heart or adrenals. More particularly, cancers include solid tumor, sarcoma, carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendothelio sarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma, retinoblastoma, a blood-born tumor, acute lymphoblastic leukemia, acute lymphoblastic B-cell leukemia, acute lymphoblastic T-cell leukemia, acute myeloblastic leukemia, acute promyelocytic leukemia, acute monoblastic leukemia, acute erythroleukemic leukemia, acute megakaryoblastic leukemia, acute myelomonocytic leukemia, acutenonlymphocyctic leukemia, acute undifferentiated leukemia, chronic myelocytic leukemia, chronic lymphocytic leukemia, hairy cell leukemia, or multiple myeloma. Particular embodiments of cancer include glioblastoma and colorectal cancer.

In one embodiment, a polypeptide comprising an amino acid sequence with at least 95% sequence identity to the amino acid sequence of SEQ ID NO: 1 (TP5) is administered. In some examples, the polypeptide comprises the amino acid sequence of SEQ ID NO: 1. In other examples, the polypeptide consists of the amino acid sequence of SEQ ID NO: 1.

The polypeptide can be modified by one or more (e.g., 1, 2, 3, 4, 5, 6, 7, 8, 9, 10, or more) amino acid substitutions, insertions or deletions, and modifications, for example, to reduce antigenicity of the polypeptide, to enhance the stability of the polypeptide and/or to improve the pharmacokinetics of the polypeptide.

Various modifications to reduce immunogenicity and/or improve the half-life of therapeutic proteins are known in the art. For example, the polypeptide can undergo glycosylation, isomerization, or deglycosylation according to standard methods known in the art. Similarly, the polypeptides can be modified by non-naturally occurring covalent modification for example by addition of polyethylene glycol moieties (pegylation) or lipidation. In one example, the compositions are conjugated to polyethylene glycol to improve their pharmacokinetic profiles.

Optionally, the polypeptide further includes a series of consecutive amino acids encoding a domain (a protein tag; for example, a myc- or his-tag) that facilitates the isolation and purification of the polypeptide.

The polypeptide (protein) can be prepared by any method, such as by synthesizing the polypeptide or by expressing a nucleic acid molecule encoding an appropriate amino acid sequence for the polypeptide in a cell and, in some embodiments, harvesting the polypeptide from the cell. A combination of such methods of production of polypeptides also can be used. Methods of de novo synthesizing peptides and methods of recombinantly producing polypeptides are known in the art (see, e.g., Chan et al., Fmoc Solid Phase Peptide Synthesis, Oxford University Press, Oxford, United Kingdom, 2005; Peptide and Protein Drug Analysis, ed. Reid, R., Marcel Dekker, Inc., 2000; Epitope Mapping, ed. Westwood et al., Oxford University Press, Oxford, United Kingdom, 2000; Sambrook et al., Molecular Cloning: A Laboratory Manual, 3rd ed., Cold Spring Harbor Press, Cold Spring Harbor, N.Y. 2001; and Ausubel et al., Current Protocols in Molecular Biology, Greene Publishing Associates and John Wiley & Sons, N Y, 1994).

The polypeptide can be labeled (e.g., to assist with the detection of the polypeptide). A “detectable label” is a molecule or material that can be used to produce a detectable signal that indicates the presence or concentration of the polypeptide or composition in a sample. Thus, a labeled polypeptide or composition provides an indicator of the presence or concentration of such in a sample. The disclosure is not limited to the use of particular labels, although examples are provided.

Any label can be employed that allows for polypeptide detection without interfering with the delivery or activity of the polypeptide. A label associated with the polypeptide or composition can be detected either directly or indirectly. A label can be detected by any mechanism including absorption, emission and/or scattering of a photon (including radio frequency, microwave frequency, infrared frequency, visible frequency and ultra-violet frequency photons). Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected by antibody binding interactions, and paramagnetic and magnetic molecules or materials. Particular examples of detectable labels include fluorescent molecules (or fluorochromes).

A fluorescent label can be a fluorescent nanoparticle, such as a semiconductor nanocrystal. Semiconductor nanocrystals are microscopic particles having size-dependent optical and/or electrical properties. When semiconductor nanocrystals are illuminated with a primary energy source, a secondary emission of energy occurs of a frequency that corresponds to the bandgap of the semiconductor material used in the semiconductor nanocrystal. This emission can be detected as colored light of a specific wavelength or fluorescence.

Additional labels include, for example, radioisotopes (such as ³H), metal chelates such as DOTA and DPTA chelates of radioactive or paramagnetic metal ions like Gd³⁺, and liposomes.

Detectable labels that can be used with the polypeptides and compositions also include enzymes, for example horseradish peroxidase, alkaline phosphatase, acid phosphatase, glucose oxidase, β-galactosidase, β-glucuronidase or β-lactamase.

Where the detectable label includes an enzyme, a chromogen, fluorogenic compound, or luminogenic compound can be used in combination with the enzyme to generate a detectable signal. Particular examples of chromogenic compounds include diaminobenzidine (DAB), 4-nitrophenylphospate (pNPP), fast red, bromochloroindolyl phosphate (BCIP), nitro blue tetrazolium (NBT), BCIP/NBT, fast red, AP Orange, AP blue, tetramethylbenzidine (TMB), 2,2′-azino-di-[3-ethylbenzothiazoline sulphonate] (ABTS), o-dianisidine, 4-chloronaphthol (4-CN), nitrophenyl-β-D-galactopyranoside (ONPG), o-phenylenediamine (OPD), 5-bromo-4-chloro-3-indolyl-β-galactopyranoside (X-Gal), methylumbelliferyl-β-D-galactopyranoside (MU-Gal), p-nitrophenyl-α-D-galactopyranoside (PNP), 5-bromo-4-chloro-3-indolyl-β-D-glucuronide (X-Gluc), 3-amino-9-ethyl carbazol (AEC), fuchsin, iodonitrotetrazolium (INT), tetrazolium blue and tetrazolium violet.

In some embodiments, the polypeptide can include at least one spacer/linker moiety. Depending on such factors as the molecules to be linked, and the conditions in which the polypeptide is being administered, the linker can vary in length and composition for optimizing such properties as flexibility and stability. In some examples, a linker is a peptide such as poly-lysine, poly-glutamine, poly-glycine, poly-proline or any combination thereof. In some examples, the peptide linker can be designed to be either hydrophilic or hydrophobic in order to enhance the activity of the polypeptide. The peptide linker and polypeptide can be encoded as a single fusion polypeptide such that the peptide linker and the polypeptide are joined by peptide bonds.

In some examples, the linker acts as a molecular bridge to link the polypeptide to a detectable label. The linker or spacer can serve, for example, simply as a convenient way to link the two entities, as a means to spatially separate the two entities, to provide an additional functionality to the peptide, or a combination thereof. For example, it may be desirable to spatially separate the polypeptide and the detectable label to prevent the detectable label from interfering with the activity of the polypeptide and/or vice versa. The linker can also be used to provide a stability sequence, a molecular tag, or various combinations thereof. In one example, the linker is one or more glycines, such as 2-10 or 4-6, including 2, 3, 4, 5, 6, 7, 8, 9 or 10 glycine residues.

The selected linker can be bifunctional or polyfunctional, e.g., containing at least a first reactive functionality at, or proximal to, a first end of the linker that is capable of bonding to, or being modified to bond to, the polypeptide and a second reactive functionality at, or proximal to, the opposite end of the linker that is capable of bonding to, or being modified to bond to the polypeptide. The two or more reactive functionalities can be the same (i.e., the linker is homobifunctional) or they can be different (i.e., the linker is heterobifunctional). A variety of bifunctional or polyfunctional cross-linking agents are known in the art that are suitable for use as linkers. Alternatively, these reagents can be used to add the linker to the polypeptide.

The length and composition of the linker/spacer can be varied considerably provided that it can fulfill its purpose as a molecular bridge. The length and composition of the linker are generally selected taking into consideration the intended function of the linker, and optionally other factors such as ease of synthesis, stability, resistance to certain chemical and/or temperature parameters, and biocompatibility. For example, the linker or spacer should not significantly interfere with the delivery of polypeptide, such as the delivery of the polypeptide to the brain, or with the activity of the polypeptide relating to regulating one or more signs or symptoms of cancer.

Linkers suitable for use according to the present disclosure may be branched, unbranched, saturated, or unsaturated hydrocarbon chains, including peptides as noted above. Furthermore, the linker can be attached to the polypeptide using recombinant DNA technology. Such methods are well-known in the art and details of this technology can be found, for example, in Sambrook et al., supra.

In one embodiment of the present disclosure, the linker is a branched or unbranched, saturated or unsaturated, hydrocarbon chain having from 1 to 100 carbon atoms, wherein one or more of the carbon atoms is optionally replaced by —O— or —NR-(wherein R is H, or C1 to C6 alkyl), and wherein the chain is optionally substituted on carbon with one or more substituents selected from the group of (C1-C6) alkoxy, (C3-C6) cycloalkyl, (C1-C6) alkanoyl, (C1-C6) alkanoyloxy, (C1-C6) alkoxycarbonyl, (C1-C6) alkylthio, amide, azido, cyano, nitro, halo, hydroxy, oxo (═O), carboxy, aryl, aryloxy, heteroaryl, and heteroaryloxy.

Examples of suitable linkers include, but are not limited to, peptides having a chain length of 1 to 100 atoms, and linkers derived from groups such as ethanolamine, ethylene glycol, polyethylene with a chain length of 6 to 100 carbon atoms, polyethylene glycol with 3 to 30 repeating units, phenoxyethanol, propanolamide, butylene glycol, butyleneglycolamide, propyl phenyl, and ethyl, propyl, hexyl, steryl, cetyl, and palmitoyl alkyl chains.

In one example, the linker is a branched or unbranched, saturated or unsaturated, hydrocarbon chain, having from 1 to 50 carbon atoms, wherein one or more of the carbon atoms is optionally replaced by —O— or —NR— (wherein R is as defined above), and wherein the chain is optionally substituted on carbon with one or more substituents selected from the group of (C1-C6) alkoxy, (C1-C6) alkanoyl, (C1-C6) alkanoyloxy, (C1-C6) alkoxycarbonyl, (C1-C6) alkylthio, amide, hydroxy, oxo (═O), carboxy, aryl and aryloxy.

In another example, the linker is an unbranched, saturated hydrocarbon chain having from 1 to 50 carbon atoms, wherein one or more of the carbon atoms is optionally replaced by O— or —NR— (wherein R is as defined above), and wherein the chain is optionally substituted on carbon with one or more substituents selected from the group of (C1-C6) alkoxy, (C1-C6) alkanoyl, (C1-C6) alkanoyloxy, (C1-C6) alkoxycarbonyl, (C1-C6) alkylthio, amide, hydroxy, oxo (═O), carboxy, aryl and aryloxy.

In a specific example, the linker is a peptide having a chain length of 1 to 50 atoms. In another embodiment, the linker is a peptide having a chain length of 1 to 40 atoms. As known in the art, the attachment of a linker or spacer to a peptide need not be a particular mode of attachment or reaction. Various reactions providing a product of suitable stability and biological compatibility are acceptable.

The invention also provides a nucleic acid molecule comprising a nucleic acid encoding the polypeptide, such as isolated nucleic acid molecules and vectors including such nucleic acid molecules. These nucleic acid molecules include DNA, cDNA, and RNA sequences, which encode the polypeptide of interest.

The nucleic acid molecule can encode heterologous polypeptides in addition the amino acid sequence of SEQ ID NO: 1, e.g., peptide linkers or other moieties to aid in the purification, detection (such as heterologous fluorescent protein sequences, such as green fluorescent protein and the like), and/or attachment of the peptides to a solid surface (such as GST, biotin, avidin or streptavidin).

To produce such nucleic acid molecules, nucleic acid sequences encoding the polypeptide are inserted into a suitable expression vector, such as a plasmid expression vector. Procedures for producing nucleic acid sequences encoding the polypeptides disclosed herein and for manipulating them in vitro are well known to those of skill in the art, and can be found (see for example, Sambrook et al., Molecular Cloning, a Laboratory Manual, 2nd edition, Cold Spring Harbor Press, Cold Spring Harbor, N.Y., 1989, and Ausubel et al., Current Protocols in Molecular Biology, Greene Publishing Associates and John Wiley & Sons, New York, N.Y., 1994).

A wide variety of cloning and in vitro amplification methodologies are known. A nucleic acid molecule encoding the polypeptide can be cloned or amplified by in vitro methods, such as the polymerase chain reaction (PCR), the ligase chain reaction (LCR), the transcription-based amplification system (TAS), the self-sustained sequence replication system (3SR) and the Qβ replicase amplification system (QB). Methods for the manipulation and insertion of the nucleic acids of this disclosure into vectors are well known in the art (see for example, Sambrook et al., Molecular Cloning, a Laboratory Manual, 2nd edition, Cold Spring Harbor Press, Cold Spring Harbor, N.Y., 1989, and Ausubel et al., Current Protocols in Molecular Biology, Greene Publishing Associates and John Wiley & Sons, New York, N.Y., 1994).

A nucleic acid molecule encoding the polypeptide can be operatively linked to expression control sequences. An expression control sequence operatively linked to a coding sequence is ligated such that expression of the coding sequence is achieved under conditions compatible with the expression control sequences. The expression control sequences include, but are not limited to, appropriate promoters, enhancers, transcription terminators, a start codon (ATG) in front of a protein-encoding gene, splicing signal for introns, maintenance of the correct reading frame of that gene to permit proper translation of mRNA, and stop codons. A promoter is an array of nucleic acid control sequences that directs transcription of a nucleic acid. A promoter includes necessary nucleic acid sequences (which can be) near the start site of transcription, such as in the case of a polymerase II type promoter (a TATA element). A promoter also can include distal enhancer or repressor elements, which can be located as much as several thousand base pairs from the start site of transcription. Both constitutive and inducible promoters are included (see, for example, Bitter et al., Methods in Enzymology 153:516-544, 1987).

The nucleic acid molecule can be incorporated into a vector into an autonomously replicating plasmid or virus or into the genomic DNA of a prokaryote or eukaryote, or which exists as a separate molecule (for example a cDNA) independent of other sequences. Typically, the nucleic acid molecules encoding the polypeptides are plasmids. However, other vectors (for example, viral vectors, phage, cosmids, etc.) can be utilized to replicate the nucleic acid molecules. In the context of this disclosure, the nucleic acid molecules typically are expression vectors (for example, prokaryotic, eukaryotic, or mammalian expression vectors) that contain a promoter sequence, which facilitates the efficient transcription of the inserted genetic sequence of the host. The expression vector typically contains an origin of replication, a promoter, as well as specific nucleic acid sequences (encoding, for example, a selectable marker) that allow phenotypic selection of the transformed cells.

Nucleic acid molecules encoding the polypeptides can be expressed in vitro by transfer into a suitable host cell. Thus, also disclosed are host cells that comprise the nucleic acid molecules and/or vectors comprising the nucleic acid molecules. The cell may be prokaryotic or eukaryotic. The term also includes any progeny of the subject host cell. It is understood that all progeny may not be identical to the parental cell since there may be mutations that occur during replication. Methods of stable transfer, meaning that the foreign nucleic acid molecule is continuously maintained in the host, are known in the art.

Transformation of a host cell with recombinant DNA can be carried out by conventional techniques as are well known to those skilled in the art. Where the host is prokaryotic, such as E. coli, competent cells, which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired, or by electroporation.

When the host is a eukaryote, methods of transfection of DNA such as calcium phosphate precipitation, conventional mechanical procedures such as microinjection, electroporation, insertion of a plasmid encased in liposomes, or virus vectors can be used. Eukaryotic cells can also be co-transformed with polynucleotide sequences encoding the polypeptide of interest, and a second foreign DNA molecule encoding a selectable phenotype (selectable marker). Another method is to use a viral vector to transiently infect or transform eukaryotic cells and express the polypeptide.

The invention further provides a vector comprising the nucleic acid molecule. Examples of suitable vectors include plasmids (e.g., DNA plasmids), yeast, listeria, and viral vectors, such as poxvirus, retrovirus, adenovirus, adeno-associated virus, herpes virus, polio virus, alphavirus, baculorvirus, and Sindbis virus.

In a first embodiment, the vector is a plasmid (e.g., DNA plasmid). The plasmid can be complexed with chitosan.

In a second embodiment, the vector is a poxvirus (e.g., chordopox virus vectors and entomopox virus vectors). Suitable poxviruses include orthopox, avipox, parapox, yatapox, and molluscipox, raccoon pox, rabbit pox, capripox (e.g., sheep pox), leporipox, and suipox (e.g., swinepox). Examples of avipox viruses include fowlpox, pigeonpox, canarypox, such as ALVAC, mynahpox, uncopox, quailpox, peacockpox, penguinpox, sparrowpox, starlingpox, and turkeypox. Examples of orthopox viruses include smallpox (also known as variola), cowpox, monkeypox, vaccinia, ectromelia, camelpox, raccoonpox, and derivatives thereof.

The term “vaccinia virus” refers to both the wild-type vaccinia virus and any of the various attenuated strains or isolates subsequently isolated including, for example, modified vaccinia Ankara (MVA), NYVAC, TROYVAC, Dry-Vax (also known as vaccinia virus-Wyeth), PDXVAC-TC (Schering-Plough Corporation), vaccinia virus-Western Reserve, vaccinia virus-EM63, vaccinia virus-Lister, vaccinia virus-New York City Board of Health, vaccinia virus-Temple of Heaven, vaccinia virus-Copenhagen, ACAM1000, ACAM2000, and modified vaccinia virus Ankara-Bavarian Nordic (“MVA-BN”).

In certain embodiments, the MVA is selected from the group consisting of MVA-572, deposited at the European Collection of Animal Cell Cultures (“ECACC”), Health Protection Agency, Microbiology Services, Porton Down, Salisbury SP4 0JG, United Kingdom (“UK”), under the deposit number ECACC 94012707 on Jan. 27, 1994; MVA-575, deposited at the ECACC under deposit number ECACC 00120707 on Dec. 7, 2000; MVA-Bavarian Nordic (“MVA-BN”), deposited at the ECACC under deposit number V00080038 on Aug. 30, 2000; and derivatives of MVA-BN. Additional exemplary poxvirus vectors are described in U.S. Pat. No. 7,211,432.

The vaccinia virus MVA was generated by 516 serial passages on chicken embryo fibroblasts of the Ankara strain of Vaccinia virus, referred to as chorioallantois virus Ankara (CVA) (see Mayr et al., Infection, 3: 6-14 (1975)). The genome of the resulting attenuated MVA lacks approximately 31 kilobase pairs of genomic DNA compared to the parental CVA strain and is highly host-cell restricted to avian cells (see Meyer et al., J. Gen. Virol., 72: 1031-1038 (1991)). It was shown in a variety of animal models that the resulting MVA was significantly avirulent (Mayr et al., Dev. Biol. Stand., 41: 225-34 (1978)). This MVA strain has been tested in clinical trials as a vaccine to immunize against smallpox in humans (see Mary et al., Zbl. Bakt. Hyg. I, Abt. Org. B, 167: 375-390 (1987); and Stickl et al., Dtsch. Med. Wschr., 99: 2386-2392 (1974)). Those studies involved over 120,000 humans, including high-risk patients, and proved that compared to vaccinia virus-based vaccines, MVA had diminished virulence or infectiousness while still able to induce a good specific immune response. Although MVA-BN is preferred for its better safety profile because it is less replication competent than other MVA strains, all MVAs are suitable for this invention, including MVA-BN and its derivatives.

Both MVA and MVA-BN are able to efficiently replicate their DNA in mammalian cells even though they are avirulent. This trait is the result of losing two important host range genes among at least 25 additional mutations and deletions that occurred during its passages through chicken embryo fibroblasts (see Meyer et al., Gen. Virol., 72: 1031-1038 (1991); and Antoine et al., Virol., 244: 365-396 (1998)). In contrast to the attenuated Copenhagen strain (NYVAC) and host range restricted avipox (ALVAC), both-early and late transcription in MVA are unimpaired, which allows for continuous gene expression throughout the viral life cycle (see Sutter et al., Proc. Nat'l Acad. Sci. USA, 89: 10847-10851 (1992)). In addition, MVA can be used in conditions of pre-existing poxvirus immunity (Ramirez et al., J. Virol., 74: 7651-7655 (2000)).

Both MVA and MVA-BN lack approximately 15% (31 kb from six regions) of the genome compared with the ancestral chorioallantois vaccinia virus Ankara (“CVA”). The deletions affect a number of virulence and host range genes, as well as the gene for Type A inclusion bodies. MVA-BN can attach to and enter human cells where virally-encoded genes are expressed very efficiently. However, assembly and release of progeny virus does not occur. MVA-BN is strongly adapted to primary chicken embryo fibroblast (CEF) cells and does not replicate in human cells. In human cells, viral genes are expressed, and no infectious virus is produced. Despite its high attenuation and reduced virulence, in preclinical studies, MVA-BN has been shown to elicit both humoral and cellular immune responses to vaccinia and to heterologous gene products encoded by genes cloned into the MVA genome (see Harrer et al., Antivir. Ther., 10(2): 285-300 (2005); Cosma et al., Vaccine, 22(1): 21-29 (2003); Di Nicola et al., Hum. Gene Ther., 14(14): 1347-1360 (2003); and Di Nicola et al., Clin. Cancer Res., 10(16): 5381-5390 (2004)).

The reproductive replication of a virus is typically expressed by the amplification ratio. The term “amplification ratio” refers to the ratio of virus produced from an infected cell (“output”) to the amount originally used to infect the cells in the first place (“input”). An amplification ratio of “1” defines an amplification status in which the amount of virus produced from infected cells is the same as the amount initially used to infect the cells, which means that the infected cells are permissive for virus infection and reproduction. An amplification ratio of less than 1 means that infected cells produce less virus than the amount used to infect the cells in the first place, and indicates that the virus lacks the capability of reproductive replication, which is a measure of virus attenuation.

Thus, the term “not capable of reproductive replication” means that an MVA or MVA derivative has an amplification ratio of less than 1 in one or more human cell lines, such as, for example, the human embryonic kidney 293 cell line (HEK293, which is deposited under deposit number ECACC No. 85120602), the human bone osteosarcoma cell line 143B (deposited under deposit number ECACC No. 91112502), the human cervix adenocarcinoma cell line HeLa (deposited at the American Type Culture Collection (ATTC) under deposit number ATCC No. CCL-2), and the human keratinocyte cell line HaCat (see Boukamp et al., J. Cell Biol., 106(3): 761-71 (1988)).

MVA-BN does not reproductively replicate in the human cell lines HEK293, 143B, HeLa, and HaCat (see U.S. Pat. Nos. 6,761,893 and 6,193,752, and International Patent Application Publication No. WO 2002/042480). For example, in one exemplary experiment, MVA-BN exhibited an amplification ratio of 0.05 to 0.2 in HEK293 cells, an amplification ratio of 0.0 to 0.6 in 143B cells, an amplification ratio of 0.04 to 0.8 in HeLa cells, and an amplification ratio of 0.02 to 0.8 in HaCat cells. Thus, MVA-BN does not reproductively replicate in any of the human cell lines HEK293, 143B, HeLa, and HaCat. In contrast, the amplification ratio of MVA-BN is greater than 1 in primary cultures of chicken embryo fibroblast cells (CEF) and in baby hamster kidney cells (BHK, which is deposited under deposit number ATCC No. CRL-1632). Therefore MVA-BN can easily be propagated and amplified in CEF primary cultures with an amplification ratio above 500, and in BHK cells with an amplification ratio above 50.

As noted above, all MVAs are suitable for this invention, including MVA-BN and its derivatives. The term “derivatives” refers to viruses showing essentially the same replication characteristics as the strain deposited with ECACC on Aug. 30, 2000, under deposit number ECACC No. V00080038 but showing differences in one or more parts of its genome. Viruses having the same “replication characteristics” as the deposited virus are viruses that replicate with similar amplification ratios as the deposited strain in CEF cells, in BHK cells, and in the human cell lines HEK293, 143B, HeLa, and HaCat.

When the vector is for administration to a subject (e.g., human), the vector (e.g., poxvirus) preferably has a low replicative efficiency in a target cell (e.g., no more than about 1 progeny per cell or, more preferably, no more than 0.1 progeny per cell are produced). Replication efficiency can readily be determined empirically by determining the virus titer after infection of the target cell.

In the case of a viral vector, the nucleic acid molecule encoding the polypeptide, as well as any other exogenous gene(s), preferably are inserted into a site or region (insertion region) in the vector (e.g., poxvirus) that does not affect virus viability of the resultant recombinant virus. Such regions can be readily identified by testing segments of virus DNA for regions that allow recombinant formation without seriously affecting virus viability of the recombinant virus.

The thymidine kinase (TK) gene is an insertion region that can readily be used and is present in many viruses. In particular, the TK gene has been found in all examined poxvirus genomes. Additional suitable insertion sites are described in International Patent Application Publication WO 2005/048957. For example, in fowlpox, insertion regions include, but are not limited to, the BamHI J fragment, EcoRI-HindIII fragment, BamHI fragment, EcoRV-HindIII fragment, long unique sequence (LUS) insertion sites (e.g., FPV006/FPV007 and FPV254/FPV255), FP14 insertion site (FPV060/FPV061), and 43K insertion site (FPV107/FPV108). In vaccinia, insertion sites include, but are not limited to, 44/45, 49/50, and 124/125.

When the vector is a recombinant fowlpox virus comprising a nucleic acid encoding the peptide and/or other exogenous gene(s) (e.g., encoding one or more immunostimulatory/regulatory molecules), the nucleic acid encoding the peptide can be inserted in one region (e.g., the FP14 region), and the exogenous gene(s) can be inserted in another region (e.g., the BamHI J region).

The inventive vector can include suitable promoters and regulatory elements, such as a transcriptional regulatory element or an enhancer. Suitable promoters include the SV40 early promoter, an RSV promoter, the retrovirus LTR, the adenovirus major late promoter, the human CMV immediate early I promoter, and various poxvirus promoters, such as the Pr7.5K promoter, 30K promoter, 40K promoter, 13 promoter, Prs promoter, PrsSynIIm promoter, PrLE1 promoter, synthetic early/late (sE/L) promoter, HH promoter, 11K promoter, and Pi promoter. While the promoters typically will be constitutive promoters, inducible promoters also can be used in the inventive vectors. Such inducible systems allow regulation of gene expression.

In one embodiment of the invention, a cell comprising (1) the polypeptide, (2) a nucleic acid molecule encoding the polypeptide, and/or (3) a vector comprising the nucleic acid molecule also is provided herein. Suitable cells include prokaryotic and eukaryotic cells, e.g., mammalian cells, yeast, fungi other than yeast, and bacteria (such as E. coli). The cell can be used in vitro, such as for research or for production of the peptide or polypeptide, or the cell can be used in vivo. In one embodiment, the cell is a yeast cell, which may be used to provide a yeast vehicle component of the yeast-based immunotherapy composition as described herein. In another embodiment, the cell can be a peptide-pulsed antigen presenting cell. Suitable antigen presenting cells include, but are not limited to, dendritic cells, B lymphocytes, monocytes, macrophages, and the like.

The polypeptide, nucleic acid molecule, vector, or cell can be isolated. The term “isolated” as used herein encompasses compounds or compositions that have been removed from a biological environment (e.g., a cell, tissue, culture medium, body fluid, etc.) or otherwise increased in purity to any degree (e.g., isolated from a synthesis medium). Isolated compounds and compositions, thus, can be synthetic or naturally produced.

The polypeptide, nucleic acid molecule, vector, or cell can be formulated as a composition (e.g., pharmaceutical composition) comprising the polypeptide, nucleic acid molecule, vector, or cell and a carrier (e.g., a pharmaceutically or physiologically acceptable carrier). Furthermore, the polypeptide, nucleic acid molecule, vector, cell, or composition of the invention can be used in the methods described herein alone or as part of a pharmaceutical formulation.

The composition (e.g., pharmaceutical composition) can comprise more than one polypeptide, nucleic acid molecule, vector, or cell of the invention. Vectors and compositions of the invention can further include or can be administered with (concurrently, sequentially, or intermittently with) any other agents or compositions or protocols that are useful for preventing or treating cancer or any compounds that treat or ameliorate any symptom of cancer. For example, the composition can comprise one or more other pharmaceutically active agents or drugs. Examples of such other pharmaceutically active agents or drugs that may be suitable for use in the pharmaceutical composition include anticancer agents (e.g., chemotherapeutic or radiotherapeutic agents), antimetabolites, hormones, hormone antagonists, antibiotics, antiviral drugs, antifungal drugs, cyclophosphamide, and combinations thereof. Suitable anticancer agents include, without limitation, alkylating agents, folate antagonists, purine antagonists, pyrimidine antagonists, spindle poisons, topoisomerase inhibitors, apoptosis inducing agents, angiogenesis inhibitors, podophyllotoxins, nitrosoureas, cisplatin, carboplatin, interferon, asparginase, tamoxifen, leuprolide, flutamide, megestrol, mitomycin, bleomycin, doxorubicin, irinotecan, taxol, geldanamycin (e.g., 17-AAG), and various anti-cancer peptides and antibodies known in the art.

Exemplary alkylating agents include, but are not limited to, nitrogen mustards (e.g., mechlorethamine, cyclophosphamide, melphalan, uracil mustard, or chlorambucil), alkyl sulfonates (e.g., busulfan), nitrosoureas (e.g., carmustine, lomustine, semustine, streptozocin, or dacarbazine). Exemplary antimetabolites include, but are not limited to, folic acid analogs (e.g., methotrexate), pyrimidine analogs (e.g., 5-fluorouracil (5-FU) or cytarabine), and purine analogs (e.g., mercaptopurine or thioguanine). Exemplary hormones and hormone antagonists include, but are not limited to, adrenocorticosteroids (e.g., prednisone), progestins (e.g., hydroxyprogesterone caproate, medroxyprogesterone acetate, and magestrol acetate), estrogens (e.g., diethylstilbestrol and ethinyl estradiol), antiestrogens (e.g., tamoxifen), and androgens (e.g., testosterone proprionate and fluoxymesterone). Other exemplary agents include, but are not limited to, vinca alkaloids (e.g., vinblastine, vincristine, or vindesine), epipodophyllotoxins (e.g., etoposide or teniposide), antibiotics (e.g., dactinomycin, daunorubicin, doxorubicin, bleomycin, plicamycin, or mitocycin C), enzymes (e.g., L-asparaginase), platinum coordination complexes (e.g., cis-diamine-dichloroplatinum II also known as cisplatin), substituted ureas (e.g., hydroxyurea), methyl hydrazine derivatives (e.g., procarbazine), and adrenocortical suppressants (e.g., mitotane and aminoglutethimide).

Chemotherapeutics that can be concurrently, sequentially or intermittently administered with the polypeptide, nucleic acid molecule, vector, cell, and/or composition disclosed herein include Adriamycin, Alkeran, Ara-C, Busulfan, CCNU, Carboplatinum, Cisplatinum, Cytoxan, Daunorubicin, DTIC, 5-FU, Fludarabine, Hydrea, Idarubicin, Ifosfamide, Methotrexate, Mithramycin, Mitomycin, Mitoxantrone, Nitrogen Mustard, Taxol (or other taxanes, such as docetaxel), Velban, Vincristine, VP-16, Gemcitabine (Gemzar), Herceptin, Irinotecan (Camptosar, CPT-11), Leustatin, Navelbine, Rituxan STI-571, Taxotere, Topotecan (Hycamtin), Xeloda (Capecitabine), Zevelin, Enzalutamide (MDV-3100 or XTANDI™), and calcitriol. Exemplary immunomodulators and/or cytokines include, but are not limited to, AS-101 (Wyeth-Ayerst Labs.), bropirimine (Upjohn), gamma interferon (Genentech), GM-CSF (granulocyte macrophage colony stimulating factor; Genetics Institute), IL-2 (Cetus or Hoffman-LaRoche), human immune globulin (Cutter Biological), IMREG (from Imreg of New Orleans, La.), SK&F 106528, tumor necrosis factor (TNF)-α, and TNF-β.

Other agents, compositions or protocols (e.g., therapeutic protocols) that are useful for the treatment of cancer in conjunction with the polypeptides (proteins), nucleic acid molecules, vectors, cells, and compositions include, but are not limited to, surgical resection of a tumor, radiation therapy, allogeneic or autologous stem cell transplantation, T cell adoptive transfer, and/or targeted cancer therapies (e.g., small molecule drugs, biologics, or monoclonal antibody therapies that specifically target molecules involved in tumor growth and progression, including, but not limited to, selective estrogen receptor modulators (SERMs), aromatase inhibitors, tyrosine kinase inhibitors, serine/threonine kinase inhibitors, histone deacetylase (HDAC) inhibitors, retinoid receptor activators, apoptosis stimulators, angiogenesis inhibitors, poly (ADP-ribose) polymerase (PARP) inhibitors, or immunostimulators).

The additional active agent (e.g., chemotherapeutics agent) can be administered before, concurrently with (including simultaneously), alternating with, sequentially, or after administration with the vectors and compositions disclosed herein. In certain embodiments, one or more (e.g., 2, 3, 4, or 5) chemotherapeutic agents is administered in combination with the vectors and compositions disclosed herein. In one embodiment, the chemotherapeutic agent is 7-ethyl-10-hydroxycamptothecin (Sn38).

The additional active agent can be administered alone or in a composition. The additional active agent can be formulated by inclusion in a vector (e.g., plasmid or viral vector), in liposomes (tecemotide, which is also known as STIMUVAX™, L-BLP25, or BLP25 liposome vaccine), or in nanoparticles (e.g., VERSAMUNE™ nanotechnology).

The carrier can be any of those conventionally used and is limited only by physio-chemical considerations, such as solubility and lack of reactivity with the active compound(s), and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the active agent(s) and one which has no detrimental side effects or toxicity under the conditions of use.

The choice of carrier will be determined in part by the particular polypeptide, nucleic acid molecule, vector, cell, or composition thereof and other active agents or drugs used, as well as by the particular method used to administer the polypeptide, nucleic acid molecule, vector, cell, or composition thereof.

The inventive methods can comprise administering a therapeutically effective amount of one or more of the polypeptide, nucleic acid molecule, vector, cell, or composition thereof to a subject. The inventive peptide, polypeptide, nucleic acid molecule, vector, cell, or composition thereof is useful for preventing emergence of cancer, arresting progression of cancer or eliminating cancer. More particularly, the polypeptide, nucleic acid molecule, vector, cell, or composition thereof can be used to prevent, inhibit or delay the development of cancer, and/or to prevent, inhibit or delay tumor migration and/or tumor invasion of other tissues (metastases) and/or to generally prevent or inhibit progression of cancer in an individual. The polypeptide, nucleic acid molecule, vector, cell, or composition thereof can also be used to ameliorate at least one symptom of the cancer, such as by reducing tumor burden in the individual; inhibiting tumor growth in the individual; increasing survival of the individual; and/or preventing, inhibiting, reversing or delaying progression of the cancer in the individual. The polypeptide, nucleic acid molecule, vector, cell, or composition thereof can be used to treat a subject with any cancer.

As used herein, the terms “treatment,” “treating,” and the like refer to obtaining a desired pharmacologic and/or physiologic effect. Preferably, the effect is therapeutic, i.e., the effect partially or completely cures a disease and/or adverse symptom attributable to the disease. To this end, the inventive method can comprise administering a “therapeutically effective amount,” which refers to an amount effective, at dosages and for periods of time necessary, to achieve a desired therapeutic result. The therapeutically effective amount may vary according to factors such as the disease state, age, and weight of the individual.

Treatment comprises, but is not limited to, destroying tumor cells, reducing tumor burden, inhibiting tumor growth, reducing the size of the primary tumor, reducing the number of metastatic legions, increasing survival of the individual, delaying, inhibiting, arresting or preventing the onset or development of metastatic cancer (such as by delaying, inhibiting, arresting or preventing the onset of development of tumor migration and/or tumor invasion of tissues outside of primary cancer and/or other processes associated with metastatic progression of cancer), delaying or arresting primary cancer progression, and/or improving the general health of the individual. It will be appreciated that tumor cell death can occur without a substantial decrease in tumor size due to, for instance, the presence of supporting cells, vascularization, fibrous matrices, etc. Accordingly, while reduction in tumor size is preferred, it is not required in the treatment of cancer.

In some examples, a therapeutic effective amount is one in which one or more signs or symptoms associated with cancer is reduced or inhibited, such as by at least 10%, for example, about 15% to about 98%, about 30% to about 95%, about 40% to about 80%, about 50% to about 70%, including about 20%, about 30%, about 40%, about 50%, about 60%, about 70%, about 80%, about 90%, about 95%, about 98% or about 100%, less than activity in the absence of the polypeptide, nucleic acid molecule, vector, cell, and/or composition.

Dosages and routes of administration for the methods of treatment are known to those of skill in the art and include, but are not limited to those described herein.

The polypeptide, nucleic acid molecule, vector, cell, or composition thereof can be administered to the subject by any method. For example, the polypeptide, or nucleic acid encoding the polypeptide (e.g., as a vector) can be introduced into a cell (e.g., in a host) by any of various techniques, such as by contacting the cell with the polypeptide, the nucleic acid molecule, or a composition comprising the nucleic acid as part of a construct, as described herein, that enables the delivery and expression of the nucleic acid. Specific protocols for introducing and expressing nucleic acids in cells are known in the art (see, e.g., Sambrook et al. (eds.), supra; and Ausubel et al., supra).

Suitable methods of administering polypeptides (proteins), nucleic acids, vectors, cells, and compositions to hosts (subjects) are known in the art. The host (subject or individual) can be any suitable host, such as a mammal (e.g., a rodent, such as a mouse, rat, hamster, or guinea pig, rabbit, cat, dog, pig, goat, cow, horse, primate, or human).

For example, the polypeptide, nucleic acid molecule, or vector (e.g., recombinant poxvirus) can be administered to a host by exposure of tumor cells to the polypeptide, nucleic acid molecule, or vector ex vivo or by injection of the polypeptide, nucleic acid molecule, or vector into the host. The polypeptide, nucleic acid molecule, vector (e.g., recombinant poxvirus) or combination of vectors, cell, and composition can be directly administered (e.g., locally administered) by direct injection into the cancerous lesion or tumor or by topical application (e.g., with a pharmaceutically acceptable carrier).

The polypeptide, nucleic acid molecule, vector, cell, or composition thereof can be administered alone or in combination with adjuvants, incorporated into liposomes (as described in, e.g., U.S. Pat. Nos. 5,643,599, 5,464,630, 5,059,421, and 4,885,172), incorporated into nanoparticles (e.g., VERSAMUNE™ nanotechnology), administered with cytokines, administered with biological response modifiers (e.g., interferon, interleukin-2 (IL-2), and/or administered colony-stimulating factors (CSF, GM-CSF, and G-CSF).

Examples of suitable adjuvants include alum, aluminum salts, aluminum phosphate, aluminum hydroxide, aluminum silica, calcium phosphate, incomplete Freund's adjuvant, saponins, such as QS21 (an immunological adjuvant derived from the bark of the South American tree Quillaja saponaria Molina), monophosphoryl lipid A (MLP-A), and RIBI DETOX™ adjuvant.

The polypeptide, nucleic acid molecule, vector, cell, or composition thereof is administered to a host (e.g., mammal, such as a human) in an amount effective to decrease cell viability of cancer cells, increase apoptosis of cancer cells, and/or treat cancer. The efficacy of the polypeptide, nucleic acid molecule, vector, or cell may be determined by in vivo or in vitro parameters as are known in the art. These parameters include but are not limited to regression of tumors, inhibition of viability of cancer cells, increase in apoptosis of cancer cells, and the like.

Any suitable dose of the polypeptide, nucleic acid molecule, vector, or cell or composition thereof can be administered to a host. The appropriate dose will vary depending upon such factors as the host's age, weight, height, sex, general medical condition, previous medical history, disease progression, and tumor burden and can be determined by a clinician. For example, the polypeptide can be administered in a dose of about 0.05 mg to about 10 mg (e.g., 0.1 mg, 0.5 mg, 1 mg, 2 mg, 3 mg, 4 mg, 5 mg, 6 mg, 7 mg, 8 mg, 9 mg, and ranges therebetween) per vaccination of the host (e.g., mammal, such as a human), and preferably about 0.1 mg to about 5 mg per vaccination. Several doses (e.g., 1, 2, 3, 4, 5, 6, or more) can be provided (e.g., over a period of weeks or months).

When the vector is a viral vector, a suitable dose can include about 1×10⁵ to about 1×10¹² (e.g., 1×10⁶, 1×10⁷, 1×10⁸, 1×10⁹, 1×10¹⁰, 1×10¹¹, and ranges therebetween) plaque forming units (pfus), although a lower or higher dose can be administered to a host. For example, about 2×10⁸ pfus can be administered (e.g., in a volume of about 0.5 mL).

The inventive cells can be administered to a host in a dose of between about 1×10⁵ and 2×10¹¹ (e.g., 1×10⁶, 1×10⁷, 1×10⁸, 1×10⁹, 1×10¹⁰, and ranges therebetween) cells per infusion. The cells can be administered in, for example, one to three (e.g., one, two, or three) infusions.

As discussed above, the polypeptide, nucleic acid molecule, vector, cell, or composition thereof can be administered to a host by various routes including, but not limited to, subcutaneous, intramuscular, intradermal, intraperitoneal, intravenous, and intratumoral. When multiple administrations are given, the administrations can be at one or more sites in a host and a single dose can be administered by dividing the single dose into equal portions for administration at one, two, three, four or more sites on the individual.

Administration of the polypeptide, nucleic acid molecule, vector, cell, or composition thereof can be “prophylactic” or “therapeutic.” When provided prophylactically, the polypeptide, nucleic acid molecule, vector, cell, or composition thereof is provided in advance of tumor formation, or the detection of the development of tumors, with the goal of preventing, inhibiting or delaying the development of tumors; and/or preventing, inhibiting or delaying metastases of such tumors and/or generally preventing or inhibiting progression of cancer in an individual. The prophylactic administration of the polypeptide, nucleic acid molecule, vector, cell, or composition thereof prevents, ameliorates, or delays cancer. When provided therapeutically, the polypeptide, nucleic acid molecule, vector, cell, or composition thereof is provided at or after the diagnosis of cancer, with the goal of ameliorating the cancer, such as by reducing tumor burden in the individual; inhibiting tumor growth in the individual; increasing survival of the individual; and/or preventing, inhibiting, reversing or delaying progression of the cancer in the individual.

When the host has already been diagnosed with cancer or metastatic cancer, the polypeptide, nucleic acid molecule, vector, cell, or composition thereof can be administered in conjunction with other therapeutic treatments such as chemotherapy, surgical resection of a tumor, treatment with targeted cancer therapy, allogeneic or autologous stem cell transplantation, T cell adoptive transfer, other immunotherapies, and/or radiation.

There are a variety of suitable formulations of the pharmaceutical composition for the inventive methods. The following formulations for parenteral, subcutaneous, intravenous, intramuscular, and intraperitoneal administration are exemplary and are in no way limiting. One skilled in the art will appreciate that these routes of administering the polypeptide, nucleic acid molecule, vector, cell, or composition of the invention are known, and, although more than one route can be used to administer a particular compound, a particular route can provide a more immediate and more effective response than another route. The polypeptide, nucleic acid molecule, vector, cell, or composition readily penetrates the blood-brain barrier when peripherally administered.

Injectable formulations are among those formulations that are preferred in accordance with the present invention. The requirements for effective pharmaceutical carriers for injectable compositions are well-known to those of ordinary skill in the art (see, e.g., Pharmaceutics and Pharmacy Practice, J.B. Lippincott Company, Philadelphia, Pa., Banker and Chalmers, eds., pages 238-250 (1982), and ASHP Handbook on Injectable Drugs, Toissel, 4th ed., pages 622-630 (1986)).

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The polypeptide, nucleic acid molecule, vector, cell, or composition thereof can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, dimethylsulfoxide, glycerol ketals, such as 2,2-dimethyl-1,3-dioxolane-4-methanol, ethers, such as poly(ethylene glycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations, include petroleum, animal, vegetable, and synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-b-aminopropionates, and 2-alkyl-imidazoline quaternary ammonium salts, and (e) mixtures thereof.

Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

The invention also provides kits that can be used to treat cancer. For example, a kit is disclosed herein for preventing or inhibiting a cancer by reducing or inhibiting one or more symptoms associated with cancer in which the kit includes at least one of polypeptides, nucleic acid molecules, vectors, cells, and/or compositions. The kits can include instructional materials disclosing means of use of the polypeptides, nucleic acid molecules, vectors, cells, and/or compositions in the kit. The instructional materials can be written, in an electronic form (such as a computer diskette or compact disk) or can be visual (such as video files). In certain examples, kits include additional compounds, such as chemotherapeutic agents.

The following examples further illustrate the invention but, of course, should not be construed as in any way limiting its scope.

### Example 1

This example demonstrates that administration of TP5 can be used to treat glioblastoma.

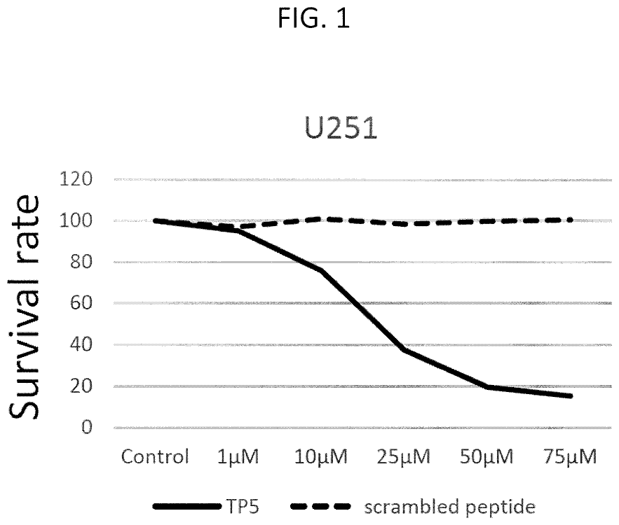

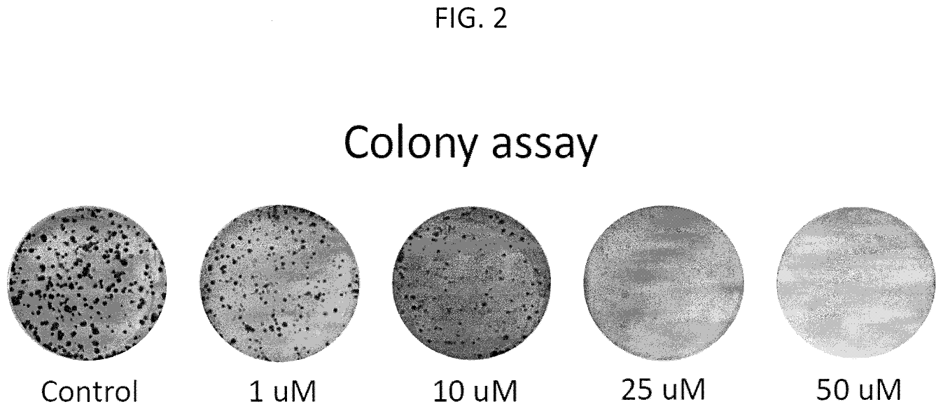

The viability of glioblastoma cell line U251 following the administration of TP5 (SEQ ID NO: 1) was determined. TP5 scrambled (SEQ ID NO: 2) was used as a control. As demonstrated by FIG. 1, TP5 but not TP5 scrambled decreased the viability of glioblastoma cell line in a dose-dependent manner. Moreover, as demonstrated by FIG. 2, TP5 decreased the number and viability of glioblastoma colonies in a dose-dependent manner.

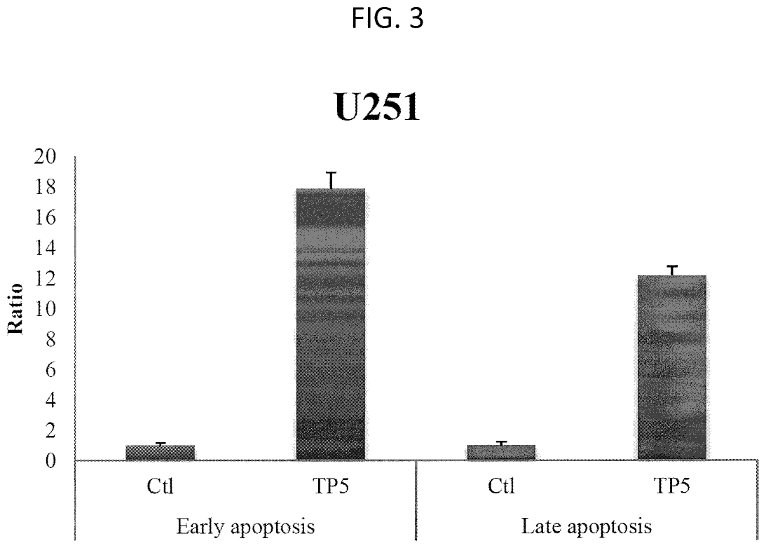

The extent of early and late apoptosis in glioblastoma cell line U251 also was determined. As indicated in FIG. 3, administration of TP5 increases the early and late apoptosis of glioblastoma cells.

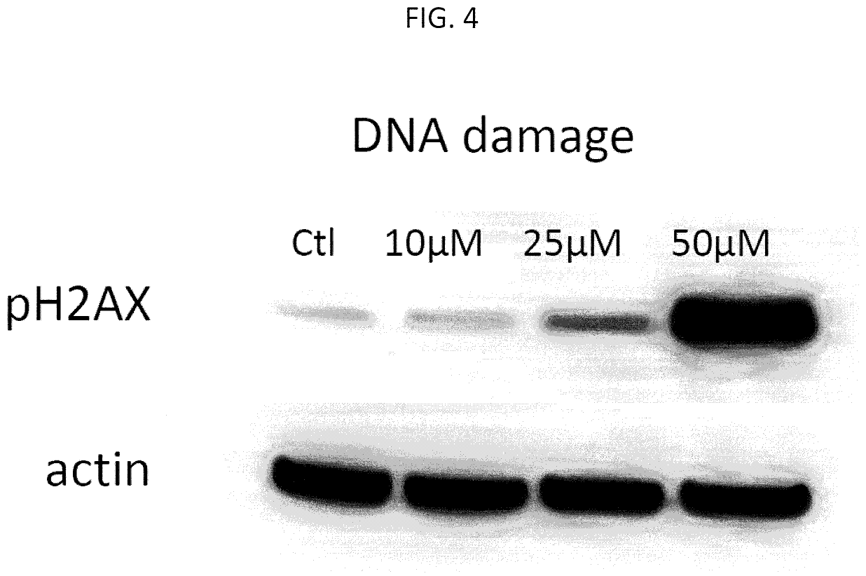

Administration of TP5 also increases DNA damage in glioblastoma cell line U251 in a dose-dependent manner as measured by expression of pH2Ax (see FIG. 4). Although not wishing to be bound by any particular theory, TP5 is believed to increase DNA damage in a dose-dependent manner and impair DNA repair by reducing G2 phase and decreasing the phosphorylation of ATM.

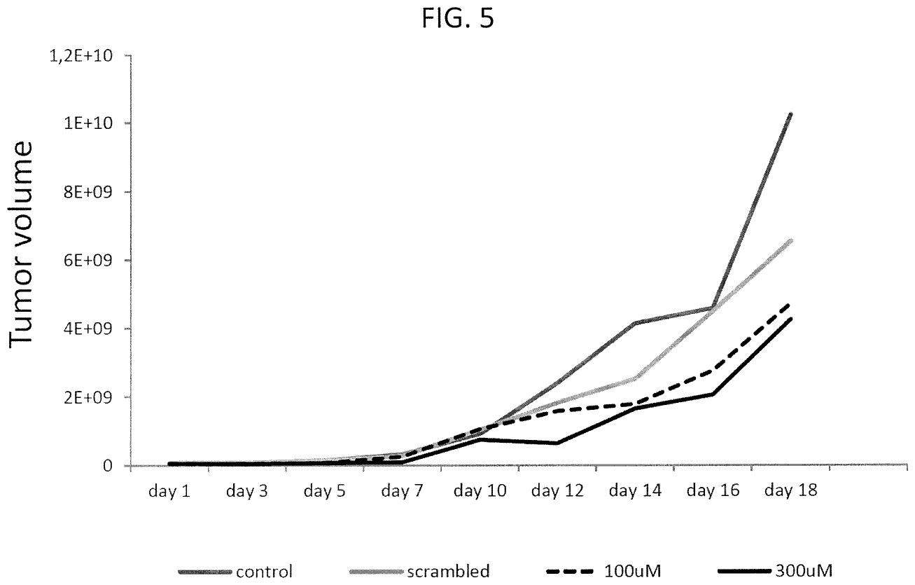

The effect of TP5 administration in an orthotopic glioblastoma mouse model additionally was determined. 100 μM or 300 μM of TP5 was administered to the mice and tumor volume and proliferation rate were investigated. TP5 but not TP5 scrambled decreases the tumor volume and proliferation rate (see FIG. 5).

### Example 2

This example demonstrates that administration of TP5 can be used to treat colorectal cancer.

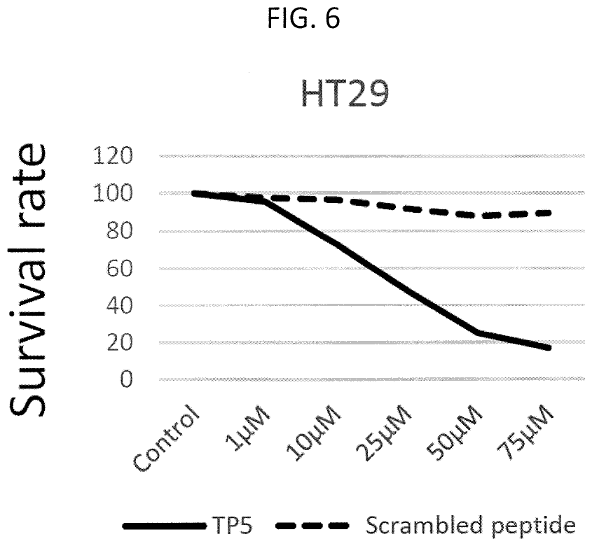

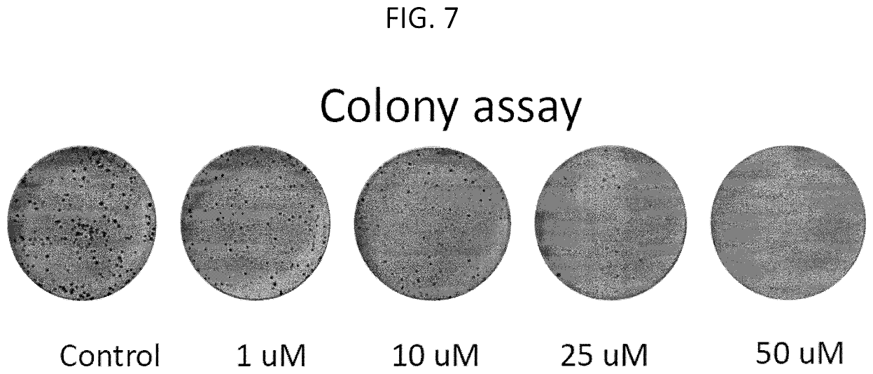

The viability of colorectal carcinoma cell line HT29 following the administration of TP5 (SEQ ID NO: 1) was determined. TP5 scrambled (SEQ ID NO: 2) was used as a control. As demonstrated by FIG. 6, TP5 but not TP5 scrambled decreased the viability of glioblastoma cell line in a dose-dependent manner. Additionally, as demonstrated by FIG. 7, TP5 decreased the number and viability of colorectal cancer colonies in a dose-dependent manner.

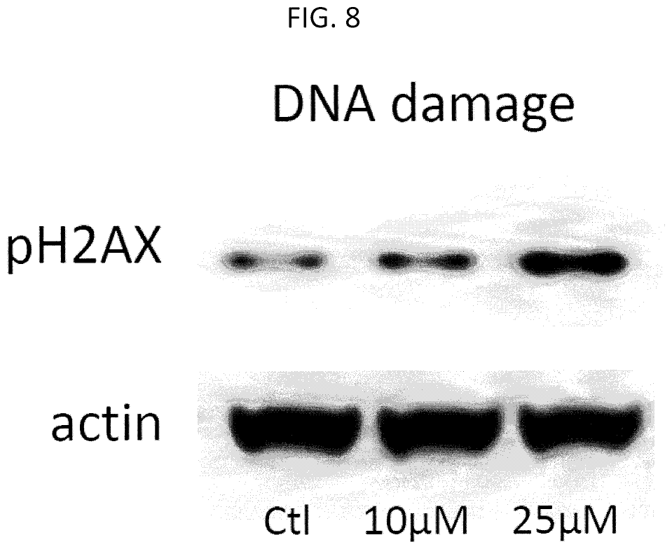

Administration of TP5 also increases DNA damage in colorectal carcinoma cell line HT29 in a dose-dependent manner as measured by expression of pH2Ax (see FIG. 8).

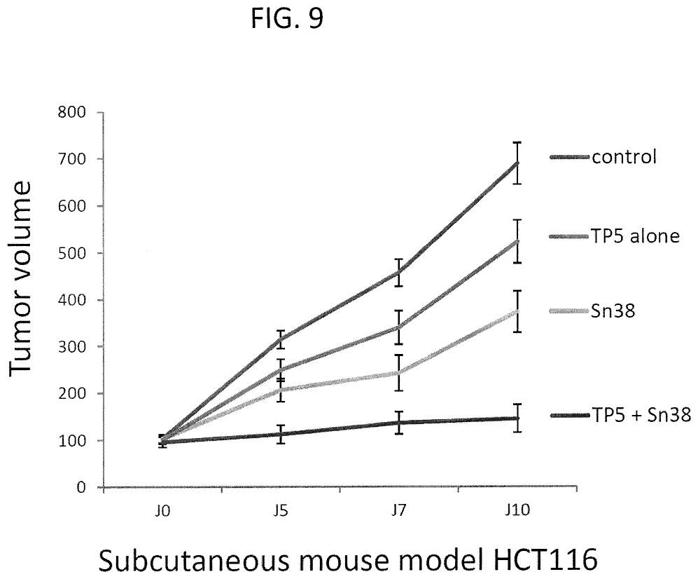

The effect of TP5 administration alone or in combination with chemotherapy (Sn38; 7-ethyl-10-hydroxycamptothecin) in a subcutaneous colorectal cancer mouse model additionally was determined. TP5, Sn38, or TP5+Sn38 was administered to the mice and tumor volume and proliferation rate were investigated. TP5 alone decreased the tumor volume of the mice. The association of TP5 and chemotherapy (Sn38) acted synergistically to decrease the tumor volume and proliferation rate (see FIG. 9).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of decreasing cell viability of cancer cells comprising administering to the cancer cells one or more of the following:
(i) a polypeptide comprising an amino acid sequence with at least 95% sequence identity to the amino acid sequence of SEQ ID NO: 1,
(ii) a nucleic acid molecule comprising a nucleic acid sequence encoding the polypeptide,
(iii) a vector comprising the nucleic acid molecule, and
(iv) a composition comprising any one of (i)-(iii).

2. A method of increasing apoptosis of cancer cells comprising administering to the cancer cells one or more of the following:
(i) a polypeptide comprising an amino acid sequence with at least 95% sequence identity to the amino acid sequence of SEQ ID NO: 1,
(ii) a nucleic acid molecule comprising a nucleic acid sequence encoding the polypeptide, and
(iii) a vector comprising the nucleic acid molecule, and
(iv) a composition comprising any one of (i)-(iii).

3. A method of treating cancer in a mammal with cancer comprising administering to the mammal one or more of the following:
(i) a polypeptide comprising an amino acid sequence with at least 95% sequence identity to the amino acid sequence of SEQ ID NO: 1,
(ii) a nucleic acid molecule comprising a nucleic acid sequence encoding the polypeptide,
(iii) a vector comprising the nucleic acid molecule,
(iv) a recombinant cell comprising any one of (i)-(iii), and
(v) a composition comprising any one of (i)-(iv).

4. The method of claim 3, wherein tumor volume in the mammal is decreased following administration of one or more of (i)-(v).

5. The method of claim 3, wherein proliferation of cancer cells in the mammal is decreased following administration of one or more of (i)-(v).

6. The method of claim 3, wherein the mammal is a human.

7. The method of claim 1, wherein the cancer is glioblastoma or colorectal cancer.

8. The method of claim 1, wherein the polypeptide comprises the amino acid sequence of SEQ ID NO: 1.

9. The method of claim 8, wherein the polypeptide consists of the amino acid sequence of SEQ ID NO: 1.

10. The method of claim 1, further comprising chemotherapy and/or radiotherapy.

11. The method of claim 1, further comprising administering a chemotherapeutic agent.

12. The method of claim 11, wherein the chemotherapeutic agent is 7-ethyl-10-hydroxycamptothecin (Sn38).

13. A (i) polypeptide comprising an amino acid sequence with at least 95% sequence identity to the amino acid sequence of SEQ ID NO: 1, (ii) nucleic acid molecule comprising a nucleic acid sequence encoding the polypeptide, (iii) vector comprising the nucleic acid molecule, or (iv) a composition comprising any one of (i)-(iii) for use in decreasing cell viability of cancer cells and/or increasing apoptosis of cancer cells.

14. A (i) polypeptide comprising an amino acid sequence with at least 95% sequence identity to the amino acid sequence of SEQ ID NO: 1, (ii) nucleic acid molecule comprising a nucleic acid sequence encoding the polypeptide, (iii) vector comprising the nucleic acid molecule, or (iv) a composition comprising any one of (i)-(iii) for use in treating cancer in a mammal with cancer.


SEQUENCE LISTING (2 sequences)

SEQ ID NO: 1 - protein, 35 aa, Artificial Sequence
  Synthetic
KEAFWDRCLSVINLMSSKMLQINAYARAARRAARR

SEQ ID NO: 2 - protein, 30 aa, Artificial Sequence
  Synthetic
FWDRCLSGKMSSKGGGINAYARAARRAARR